(19) United States
(12) Patent Application Publication
(10) Pub. No.: US 2026/0083403 A1
(43) Pub. Date: Mar. 26, 2026

(54) REINFORCED SURGICAL TABLE MATTRESSES AND PADS

(71) Applicant: Augustine Temperature Management LLC, Eden Prairie, MN (US)

(72) Inventor: Scott D. Augustine, Deephaven, MN (US)

(21) Appl. No.: 19/307,652

(22) Filed: Aug. 22, 2025

Related U.S. Application Data
(60) Provisional application No. 63/697,302, filed on Sep. 20, 2024.

(51) Int. Cl. A61B 5/00; A61B 7/00; A61G 13/12; B29C 65/00; B29C 65/54; B29C 65/72; B29L 31/00; B32B 3/02; B32B 3/04; B32B 5/02; B32B 5/18; B32B 5/24; B32B 7/12; B32B 27/12

(52) U.S. Cl. CPC A61B 5/6892; A61B 5/7264; A61B 7/003; A61G 13/126; B29C 65/54; B29C 65/72; B29C 66/61; B29C 66/727; B29C 66/729; B29C 66/73187; B32B 3/02; B32B 3/04; B32B 5/02; B32B 5/18; B32B 5/245; B32B 7/12; B32B 27/12; A61B 2562/164; B29L 2031/7546; B32B 2266/0214; B32B 2307/7265; B32B 2535/00

(57) ABSTRACT

Reinforced surgical table mattresses or pads and methods of manufacture are disclosed. A layer of polymeric foam can be inserted into a shell formed from one or more sheets of flexible polymeric film material laminated to a fabric and welded together to create a water-proof shell. At least one layer of hotmelt glue film can be positioned inside the shell between the bottom side of the foam and the fabric of the shell. Heat can be applied to the outside of the shell at a temperature above the melting point of the hotmelt glue film and below that of the shell material, melting the glue film and bonding the fabric of the shell to an adjacent layer of material when cooled.

### RELATED APPLICATIONS

This application claims priority to U.S. Provisional Application 63/697,302, filed on Sep. 20, 2024, the entire contents of which are hereby incorporated by reference.

### TECHNICAL FIELD

The present invention is related to surgical table mattresses and pads.

### BACKGROUND

Surgical table mattresses have changed very little over the last 70+ years. Then and now, they are made of a compressible foam pad that is encased in a water-proof polymeric film shell. The foam may be a single layer or multiple layers of different foam materials laminated together. The shells are typically made of a thin layer of urethane that has been laminated to a thin and stretchable layer of woven or knitted fabric for added strength. The mattresses are typically removably attached to the top of the surgical table with hook and loop (Velcro) fasteners.

When heavy patients move around on the mattress or are put into challenging positions such as steep head-down (Trendelenburg) position, considerable torque and sheer force is applied to the relatively thin shell material. The shell material is also stressed when the mattress is removed from the table-top for cleaning between cases. Since the loop side of the hook and loop fasteners is attached to the underside of the mattress shell, the force necessary to pull the hook and loop fasteners apart is applied to the shell and it is common for the shells to fail at the seams with prolonged use and removal from the table for cleaning.

While hook and loop fasteners are commonly used in the operating room, they are generally limited to disposable, single use products because they are nearly impossible to clean. Since surgical table mattresses are reusable and may be used for two years or more, cleaning the hook and loop fasteners is a problem. Another problem with the traditional hook and loop fasteners is that over time, the hooks can break off and eventually there may not be enough hooks left to secure the mattress to the table.

The operating room is a noisy environment and auscultation of subtle sounds from the patient can be impossible with the background noise.

### SUMMARY

Certain embodiments of the invention include a pad or mattress comprising a foam layer and a shell. The shell covers the pad or mattress and includes one or more sheets of flexible polymeric film material welded together at the seams. In some embodiments the weld couples the sheets together about the edges of the mattress. In some embodiments, the weld couples the sheets about the edges of the sheets. Although the pad or mattress is described as having two or more sheets welded together, as one of ordinary skill in the art would consider, the shell could be formed from one sheet folded around the foam layer and welded at the corners.

Certain embodiments of the invention include a compressible material layer comprising a foam or air mattress pad, and a shell. The foam may be a single layer or multiple layers of different foam materials laminated together. Surgical table mattress or pads are sized to fit the top of the surgical table. They are usually segmented with the largest segment being the torso pad.

In some examples, the shells are made of a thin layer of urethane film that has been laminated to a thin and stretchable layer of woven or knitted fabric for added strength. The shell covers the foam layer and includes one or more sheets of flexible polymeric material welded or sewn together.

Shells are generally constructed by welding the shell material into a shell with a slit-like opening on either the bottom side or end which allows the insertion of the foam layer. After the foam layer is inserted, the slit-like opening is sealed or welded shut or sewn and sealed with an adhesive. Inserting the foam layer into the shell can be very challenging. The shell is generally not attached to the foam layer. Introducing an adhesive between the shell and the foam layer during the assembly process to adhesively attach the foam layer to the shell, would be difficult and messy. Without adhesively attaching the shell to the foam layer, the shell cannot benefit from the mechanical reinforcement that could be provided to the shell from the foam layer.

In some examples, the relatively fragile shell material may be reinforced by adhesively bonding a layer of fabric to the inside of the shell material. The layer of fabric may be bonded to the inside of the lower layer of shell material, thus reinforcing the underside of the mattress. In some examples, the layer of fabric may be bonded to the inside of the upper layer or top layer of the shell material, thus reinforcing a portion of the top side or the entire top side of the mattress. In some examples, the layer of fabric may be bonded to both the upper and lower layer.

In some examples the fabric may also be bonded to the foam layer. In this case, adhesive would be applied to both sides of the fabric layer allowing it to be bonded to both the shell and the foam layer.

In some examples, the adhesive bonding may advantageously be accomplished with hotmelt glue. Hotmelt glue comes in many formulations using various polymeric materials, including but not limited to ethylene vinyl acetate glue (EVA). The various formulations have different melting temperatures. Hotmelt glue comes in many forms including a film. A film of glue can be easily applied to a layer of fabric by heating it above its melting temperature and then allowing it to cool. Unlike other adhesives, once it cools it is no longer sticky. Hotmelt glue is susceptible to re-melting by a hot heat-source, but is otherwise very durable, stable and flexible.

In some examples, hotmelt glue may be applied to one or both sides of the layer of fabric and allowed to cool. As a manufacturing convenience, the layer of fabric with the cooled, non-sticky glue applied to one or both sides, can then be inserted inside the shell between the shell and the foam and positioned without sticking to anything. Alternatively, the layer of fabric and the hotmelt glue layer may be adhered to the foam layer before inserting the foam layer into the shell.

In some examples, when the layer of fabric and glue have been correctly positioned inside the shell, heat can be applied to the outside of the shell, penetrating the shell and melting the glue to bond the fabric to the shell. If glue is applied to both sides of the layer of fabric, the layer of fabric would also be adhesively attached to the foam layer after heating.

In some examples, the heat to melt the hotmelt glue can advantageously be safely applied externally through the shell layer because the urethane film of the shell layer has a much higher melting temperature than the EVA hotmelt glue. Therefore, if the heating iron temperature is set to a temperature between the melting temperature of the EVA glue and the urethane shell melting temperature, the hotmelt glue will melt and the shell will suffer no damage. When the heating iron is removed, the glue will cool and securely bond the shell to the fabric and if hotmelt glue was applied to both sides of the fabric, it will be securely bonded to the foam layer as well. This technique is inexpensive, simplifies the manufacturing process, makes no sticky adhesive mess and is not visible by external inspection of the shell.

The attachment of the mattress pad to the surgical tabletop with a hook and loop fasteners (Velcro) can be problematic for three reasons. The significant purchase by the hook and loop fasteners that is needed to prevent the mattress pad from moving on the tabletop, can result in damage to the shell material when the pad is repeatedly removed from the tabletop for cleaning between surgical cases. Second, the hook and loop fasteners are nearly impossible to clean and finally, the hooks can break off with use and eventually there are not enough left to secure the pad to the tabletop.

In some examples, it might be advantageous to use a different technology than hook and loop fasteners for securing the surgical table pad or mattress to the surgical tabletop. It should be understood that the primary force applied to the surgical table pad during regular use is a sliding force, not a force trying to lift the pad from the table. In order to prevent sliding, hook and loop fasteners provide much more resistance to lifting than is necessary and that is why they can damage the shell during removal from the tabletop for cleaning.

In some examples, it may be advantageous to replace the hook and loop fasteners with four or more “post and cup” fasteners. The four “post and cup” fasteners may be located under the mattress near the sides and generally near the corners. Additional “post and cup” fasteners may be located under the mattress along the sides and between the four fasteners in the corners.

In some examples, a post and cup fastener comprises a molded plastic post component that may be 0.25-0.5 in. high and 1-3 in. in diameter. In some examples, the post also includes a surrounding flange that can be adhesively attached to the stainless-steel top of the surgical table.

In some examples, a post and cup fastener comprises a molded plastic cup component that may be the same shape and is slightly larger in diameter than the matching post component. The molded plastic cup component may be roughly the same height as the matching post component. In some examples, the cup component may be closed on the side facing the mattress shell and may or may not include a surrounding flange, all of which can be adhesively attached to the bottom side of the mattress shell.

The cup component can fit over the top of the matching post component, preventing any sliding movement of the mattress against the tabletop. The low profile of the post and cup fastener is imperceptible under the 2-4 inch thick foam compressible material layer. In some examples, a magnet may be attached under the closed surface of the post component and a flat piece of iron attached under the closed surface of the cup component, in opposing locations where the magnet and piece of iron can magnetically engage with each other through the plastic layers of the closed central sections of the post and cup. In this location, the magnet and piece of iron are hidden from view and protected from liquid contamination. The magnetic force between the magnet and piece of iron, is adequate to keep the cup component seated over the post component during normal use and is still relatively easy to disengage the cup component from the post component in order to remove the mattress from the tabletop.

An advantage of the surgical mattress securement system of this disclosure is that it can be easily retrofitted onto legacy surgical tabletops in the field. In some examples, the post components can be fitted into the matching cup components that are attached to the bottom of the mattress in the factory. The release liners covering the pressure sensitive adhesive (PSA) on the flanges of the post components can be removed and the mattress placed in its proper position on the surgical table. The post components will adhesively attach to the tabletop and will be automatically aligned with their matching cup components. The ability to easily and reliably retrofit this new technology to legacy surgical tables already installed in the operating room, provides a significant advantage for installation.

Auscultation of breath sounds and other sounds from the chest is routinely accomplished by placing a stethoscope on the patient's back, over the lungs and listening. This is difficult in the operating room because most patients are operated on in the supine (back down) position, laying on their back. Auscultation of gross breath and heart sounds from the anterior chest and/or neck has been a standard monitor during anesthesia for the past ˜100 years. With the advent of electronic monitors such a pulse oximetry and end-tidal CO₂, clinicians have relied less and less on auscultation of gross breath and heart sounds, and many have abandoned routine auscultation all together.

Operating rooms are noisy environments and auscultation of subtle sounds with a standard stethoscope is impossible due to the significant background noise and the limitations of human hearing. As a result, auscultation of any sounds other than gross breath sounds in the operating room, has been impractical.

Microphones have been added to standard stethoscopes, but they amplify all sounds including the background noise. Simple electronic amplification of auscultated sounds has not been very useful.

Most surgery is done in the supine or lithotomy position (the patients back laying on the mattress). Since operating tables are essentially the same size as the adult patient, in most cases it is predictable where the patient's chest will be located on the table.

In some examples, one or more microphones may be placed between the shell material and the foam layer of the mattress pad or on top of the shell material of the mattress pad of this disclosure and positioned where the patient's lungs are anticipated to be located on the mattress pad. With only a thin layer of shell material or plastic film and maybe a bedsheet separating the microphone from the skin of the patient's back, the one or more microphones may be perfectly positioned to receive high-quality sounds from the patient's chest, through the back.

In some examples, the 2-4 in. of foam in the foam layer of the mattress and a steel tabletop under the one or more microphones, uniquely provides excellent sound deadening of the background noise. If the background noise can be deadened, then the subtle breath sounds and other sounds emanating from the chest can be detected by the one or more microphones, amplified electronically and recorded.

In some examples, the electronically recorded breath sounds may be analyzed with artificial intelligence (AI) or machine learning (ML). In contrast to the humans ability to hear sounds that are all jumbled together as a single sound, the computer using AI can separate the jumbled sounds into their individual components. This is similar to the sound of an orchestra at a point in time sounding like a single sound to the human ear. In contrast, the computer can separate the sound into its individual components of individual instruments. Because the sounds can be recorded, AI can look for subtle changes in a given sound over time, whereas the human ear could never discern subtle changes over time.

In some examples, breath sounds may be monitored to document successful tracheal intubation and ventilation. Breath sounds may be monitored to document spontaneous ventilation. In some examples, wheezing emanating from large airways may be separated from wheezing emanating from small airways, where the treatment may be different. Breath sound symmetry between the two sides of the chest may be monitored to detect tracheal tube misplacement or a pneumothorax. Breath sounds may be monitored and used for “sensor fusion” validation of ventilation.

The sound of the heart beating may be monitored and used for “sensor fusion” validation of the electrocardiogram (ECG) trace. A loose ECG lead can make the ECG trace look like ventricular fibrillation (V-fib). V-fib can quickly be ruled out if a rhythmic “lub-dub” is heard with auscultation. Heart sounds including subtle murmurs can be monitored for changes over time indicating changes in blood flow.

In some examples, the sound of blood flowing through the major arteries such as the aorta may be correlated with cardiac output. An absolute cardiac output number may not be determinable but relative cardiac outputs over time may be determined.

It is apparent that auscultation with deadening of the background noise and AI interpretation can serve as a monitor for a wide variety of physiologic parameters, some of which are qualitative and some are quantitative.

### BRIEF DESCRIPTION OF THE DRAWINGS

The following drawings are illustrative of particular embodiments of the present invention and therefore do not limit the scope of the invention. The drawings are not to scale (unless so stated) and are intended for use in conjunction with the explanations in the following detailed description. Embodiments of the present invention will hereinafter be described in conjunction with the appended drawings, wherein like numerals denote like elements.

FIG. 1 is a cutaway perspective top view of a reinforced surgical table mattress or pad in accordance with embodiments of the invention.

FIG. 2 is a perspective bottom view of a reinforced surgical table mattress or pad in accordance with embodiments of the invention.

FIG. 3 is a cross sectional view at 22-22 of FIGS. 1 and 2, of a reinforced surgical table mattress or pad in accordance with embodiments of the invention.

FIG. 4 is a cross sectional view at 22-22 of FIGS. 1 and 2, of a reinforced surgical table mattress or pad in accordance with embodiments of the invention.

FIG. 5 is a cross sectional view at 22-22 of FIGS. 1 and 2, of a reinforced surgical table mattress or pad in accordance with embodiments of the invention.

FIG. 6 is a cross sectional view at 22-22 of FIGS. 1 and 2, of a reinforced surgical table mattress or pad in accordance with embodiments of the invention.

FIG. 7 is a cross sectional view at 22-22 of FIGS. 1 and 2, of a reinforced surgical table mattress or pad in accordance with embodiments of the invention.

FIG. 8 is a cross sectional view at 22-22 of FIGS. 1 and 2, of a reinforced surgical table mattress or pad in accordance with embodiments of the invention.

FIG. 9 is a partial cross sectional view at 30-30 of FIG. 2, of a reinforced surgical table mattress or pad with the addition of a partial cross section of a surgical tabletop in accordance with embodiments of the invention.

### DETAILED DESCRIPTION

The following detailed description is exemplary in nature and is not intended to limit the scope, applicability, or configuration of the invention in any way. Rather, the following description provides practical illustrations for implementing exemplary embodiments of the present invention. Examples of constructions, materials, dimensions, and manufacturing processes are provided for selected elements, and all other elements employ that which is known to those of skill in the field of the invention. Those skilled in the art will recognize that many of the examples provided have suitable alternatives that can be utilized. The term mattress, used to describe embodiments of the present invention, may be considered to encompass mattresses and pads, and vice-versa. Pads may also be referred to as underbody support systems or mattresses.

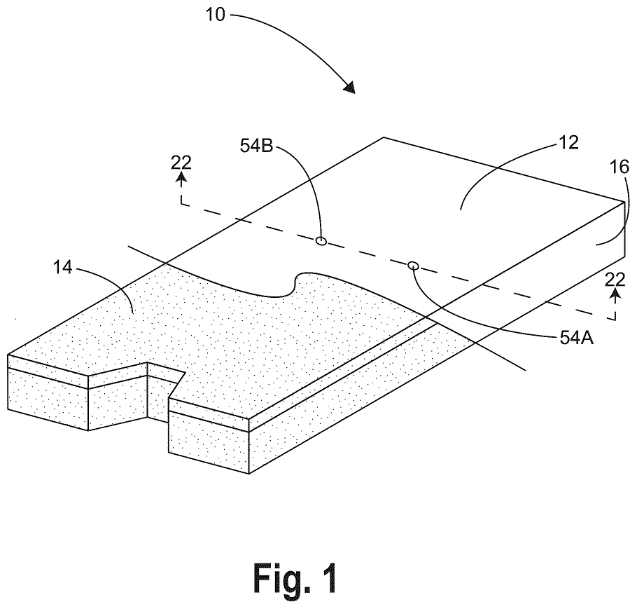

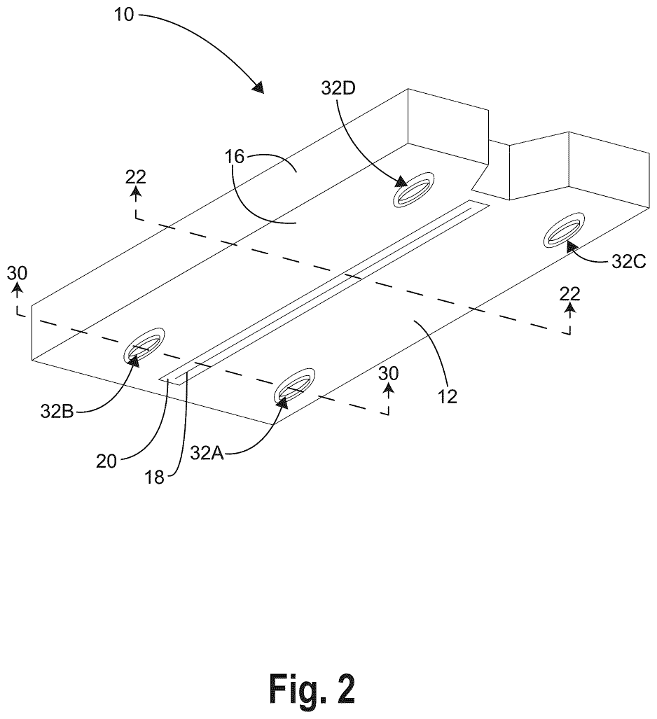

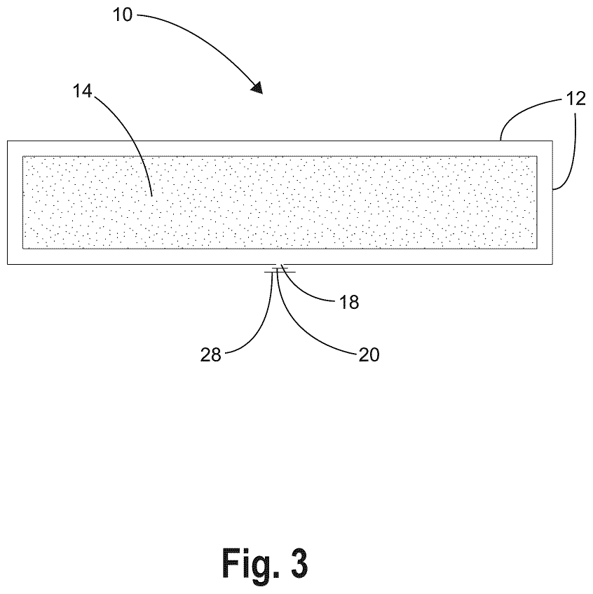

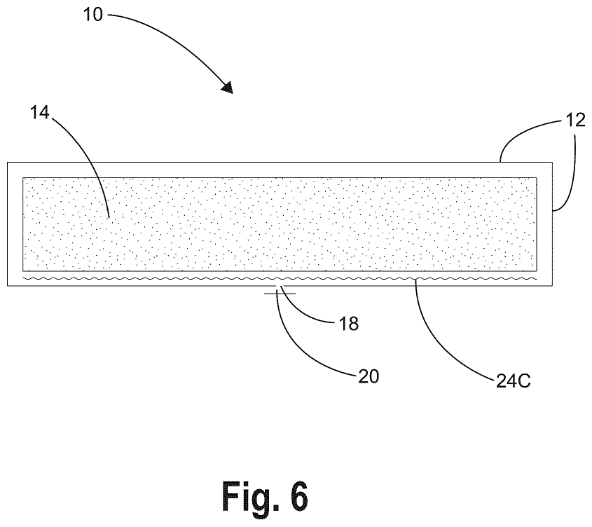

As shown in FIGS. 1-8, certain embodiments of the invention include a pad or mattress 10 comprising a foam layer 14 and a shell 12. FIGS. 3-8 are cross sections at 22-22 in FIGS. 1-2. The shell 12 covers the pad or mattress 10 and includes one or more sheets of flexible polymeric film material 16 welded together at the seams. In some embodiments the weld couples the sheets together about the edges of the mattress 10. In some embodiments, the weld couples the sheets 16 about the edges of the sheets 16. Although the pad or mattress 10 is described as having two or more sheets 16 welded together, as one of ordinary skill in the art would understand, the shell 12 could be formed from one sheet 16 folded around the foam layer 14 and welded at the corners. The relatively thin flexible polymeric film material 16 may be laminated to a sheet of woven or knit fabric for added strength.

Certain embodiments of the invention include a compressible material layer 14 comprising a foam or air mattress pad, and a shell 12. The foam layer 14 may be a single layer or multiple layers of different foam materials laminated together. Maximal pressure off-loading effectiveness is achieved by maximizing the accommodation of the patient into the mattress 10. This means that the patient should maximally sink into the foam or compressible material layer 14. In some embodiments, the compressible material layer 14 comprises one or more flexible air-filled chambers. The foam (compressible material) or air bladders of the mattress should be easily deformable to allow the patient to sink into the mattress. This accommodation maximizes the patients skin surface area in contact with the mattress 10, which minimizes the pressure applied to the skin at any given point.

As shown in FIG. 1, in some embodiments, the upper part of the compressible material layer or foam layer 14 may be a high-tech foam to reduce the pressure exerted against the patient's skin during surgery. High-tech foams include but are not limited to visco-elastic foams (“memory foams”) that are designed to maximize accommodation of the patient into the mattress or pad 10. As previously noted, accommodation refers to the sinking of the user, such as the patient, into the pad 10 until a maximal amount of support surface area is in contact with a maximal amount of skin surface, and the pressure exerted across the skin surface is as uniform as possible. These high-tech foam materials may accommodate the patient more effectively than simple urethane upholstery foam. In some examples, the lower layers of foam in the mattress or pad 10 may be one or more layers of urethane foam.

Surgical table mattresses or pads are sized to fit the top of the surgical table. They are usually segmented with the largest segment being the torso pad. Torso pads, depending on the table size, are generally approximately 20″ wide by 45″ long and 2-4″ thick, fitting the torso section of the surgical table. The pads may be wider for wider bariatric surgical tables.

In some examples, the shells 12 are made of a thin layer of urethane film that has been laminated onto a thin and stretchable layer of woven or knitted fabric, for added strength. Urethane, PVC and other flexible polymeric film materials are contemplated. Many other suitable plastic film or fiber-reinforced plastic film shell materials are contemplated. The shell 12 is preferably water resistant, flexible, and durable enough to withstand the wear and tear of operating room use. In some embodiments, the polymeric shell material is about 0.002-0.015 inch thick. In this thickness range, both urethane and PVC, for example, are strong but retain an adequate stretchability.

In some examples, the upper most layer of foam 14 may be a softer, low density urethane foam or an elastomeric memory foam and the bottom layer(s) of foam 14 may be a denser urethane foam that prevents “bottoming out.” In some examples, the mattress 10 may include a polymeric foam compressible material layer 14 that has been hollowed out to substantially form a foam box in which transverse inflatable air tubes can be housed, creating a combination foam and air mattress.

In some examples, the shell 12 covers the foam layer 14 and includes one or more sheets 16 of flexible fabric-reinforced, polymeric material welded or sewn together. The welds may be accomplished by heat, ultrasound or radio frequency welding. Alternatively, in some examples sewing and/or adhesive bonding may be used instead of welding.

The mattress 10 may cover approximately the entire surface of the surgical table or any other bed or chair such as a wheel chair. Alternately, the mattress 10 may be sized to fit some or all of the sections that form the support surface of a surgical table. For example, if the table has multiple separate sections, such as three, the mattress pads 10 may be sized to fit each of the three table sections.

As shown in FIGS. 2-3, shells 12 are generally constructed by welding the shell material 16 into a shell 12 with a slit-like opening 18 on either the bottom side or end, which allows the insertion of the foam layer 14 into the finished fully-formed shell 12. After the foam layer 14 is inserted into the shell 12, the slit-like opening 18 is sealed or welded shut. Alternatively, the slit-like opening 18 may be sewn shut and the stitch holes sealed with an adhesive. In some examples, the slit-like opening 18 is sealed shut by welding a strip of the shell polymer material 20, over the slit-like opening 18. In some examples, the weld may be a heat weld. Alternatively, the slit-like opening 18 may be closed with a solvent or adhesive bond to a strip of shell polymer material 20.

Inserting the foam layer 14 into the shell 12 during the manufacturing process can be very challenging. The shell 12 is generally not attached to the foam layer 14. The foam layer 14 often must be inserted through the slit-like opening 18 and then positioned naturally within the shell 12, avoiding wrinkles. Since the shell 12 is sized to fit snugly over the foam layer 14 and the fabric layer of the shell material 12 “sticks” to the foam layer 14, it can be very challenging to insert the foam layer 14 and naturally position it without wrinkling the shell 12 or foam layer 14. Introducing an adhesive between the shell 12 and the foam layer 14 during the assembly process to adhesively attach the foam layer 14 to the shell 12, is difficult and messy. Without adhesively attaching the shell 12 to the foam layer 14, the shell 12 cannot benefit from the mechanical reinforcement that could be provided to the shell 12 from the foam layer 14.

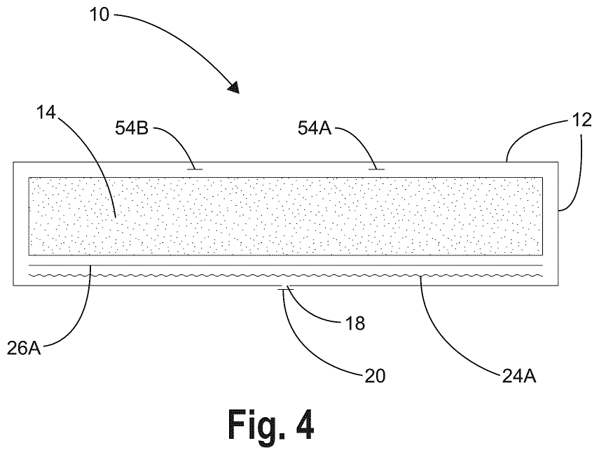

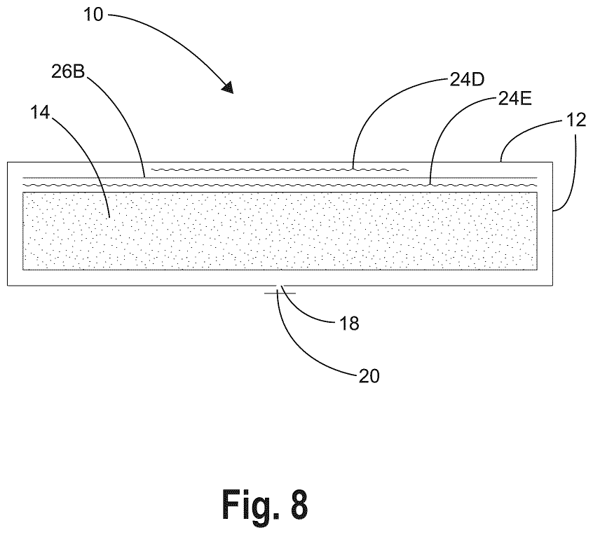

In some examples, the relatively fragile shell 12 material may be reinforced by adhesively bonding a layer of fabric 26A, B to the inside of the shell 12 material. As shown in FIGS. 4,5 the layer of fabric 26A may be bonded to the inside of the lower layer of shell 12 material, thus reinforcing the underside of the mattress 10. In some examples, as shown in FIG. 8, the layer of fabric 26B may be bonded to the inside of the layer of the shell 12 material on the top side of mattress 10, thus reinforcing a portion of the top side or the entire top side of the mattress 10. In some examples, the layers of fabric 26A, B may be bonded to the layers of shell 12 material on both the top side of mattress 10 the bottom side of mattress 10. In some examples, the layers of fabric 26A, B may extend beyond the edges or ends of the top or bottom sides of the foam layer 14 and fold onto the sides or ends where it may be bonded to the sides or ends of the foam layer 14. In this case the layers of fabric 26A, B may overlay one or more seams at the edges of the mattress 10, thus reinforcing the seam. In some examples, the fabric layers 26A, B may be a woven fabric or a non-woven fabric.

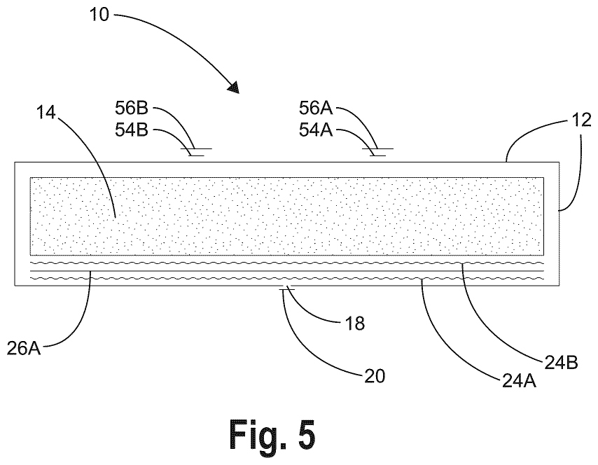

In some examples, the layers of fabric 26A, B may also be adhesively bonded 24B, E to the foam layer 14 as shown in FIGS. 5,8. In this case, adhesive 24A, B may be applied to both sides of the fabric layer 26A, B allowing it to be bonded to both the shell 12 and the foam layer 14.

Adhesively bonding the layers inside the shell 12 after the foam layer 14 has been inserted is very difficult and messy whether the adhesive is a liquid, paste or pressure sensitive adhesive (PSA). PSA sticks to everything during positioning unless covered with a release liner. Removing a release liner from inside the form-fitting shell 12 is nearly impossible. Paste and liquid adhesives are generally applied in lines or traces which can then be seen as unsightly deformities of the thin shell material 12. For the best visual result, the shell material 12 is ideally perfectly smooth. Visible lines or traces of adhesive disrupting the otherwise smooth surface of the shell 12 is not ideal.

In some examples, the adhesive bonding 24A-E may advantageously be accomplished with hotmelt glue. Hotmelt glue comes in many formulations of various polymeric materials, including but not limited to ethylene vinyl acetate glue (EVA). The various formulations have different melting temperatures. Hotmelt glue comes in many forms including a film. Other forms are contemplated in this disclosure. A film of hotmelt glue 24A-E can be easily applied to a layer of fabric 26A, B by heating it above its melting temperature and then allowing it to cool. Unlike other adhesives, once it cools it is no longer sticky. Hotmelt glue is susceptible to re-melting by a hot heat-source, but is otherwise very durable, stable and flexible. Unlike pressure sensitive adhesives (PSA), hotmelt glue will not pull apart in response to a prolonged force.

In some examples, hotmelt glue film may be between 0.002 and 0.030 in. thick. An advantage of applying hotmelt glue in film form is that the application is perfectly uniform across the entire bonded surface. Since the shell 12 material is relatively thin, any variations in glue application on the inside of the shell 12 will be visible on the outside of the shell 12. For example, if the hotmelt glue is applied in serpentine traces, the serpentine traces will be visible from the outside of the shell 12. In contrast, a film of hotmelt glue on the inside of the shell 12 will create a smooth, uniform surface visible on the outside of the shell 12. Clearly, a smooth surface appearance of the shell is visually preferable.

In some examples, hotmelt glue 24A-E may be applied to one or both sides of the layer of fabric 26A, B and allowed to cool prior to assembly of the mattress pad 10. As a manufacturing convenience, the layer of fabric 26A, B with the cooled, non-sticky glue 24A-E applied to one or both sides, can then be inserted into the shell 12 through the slit-like opening 18. The layer of fabric 26A, B with the cooled, non-sticky glue 24A-E can be positioned between the shell 12 and the foam layer 14 and smoothed out without sticking to anything. Alternatively, the layer of fabric 26A, B and the hotmelt glue layer 24A-E may be adhered to the foam layer 14 before inserting the foam layer 14 into the shell 12. In any case, the cooled hotmelt glue layer 24A-E can be inserted into the shell 12 without sticking to the shell 12 and allowing the shell 12 to be positioned naturally without wrinkles before adhering the hotmelt glue layer 24A-E by heating it to a temperature above a melting temperature of the hotmelt glue layer 24A-E and then allowing it to cool.

In some examples, when the layer of fabric 26A, B and glue layer 24A-E have been correctly positioned inside the shell 12, heat can be applied to the outside of the shell 12, penetrating the shell 12 and melting the glue layer 24A-E to bond the fabric 26A, B to the shell 12 and/or foam 14. If hotmelt glue layer 24A-E is applied to both sides of the layer of fabric 26A, B, heat can penetrate the layer of fabric 26A, B to melt the hotmelt glue facing the foam layer 14, and the layer of fabric 26A, B can be adhesively attached to the foam layer 14 after heating and allowing the hotmelt glue to cool.

In some examples, the heat to melt the hotmelt glue layer 24A-E can advantageously be safely applied externally through the shell layer 12, because the urethane film of the shell layer 12 has a much higher melting point temperature than the EVA hotmelt glue. Therefore, if the heating iron temperature is set to a temperature greater than the melting temperature of the EVA glue and less than the urethane shell melting temperature, the hotmelt glue layer 24A-E will melt and the shell 12 will suffer no damage. The heating iron can be applied directly to the urethane shell 12 material or to a protective Teflon layer temporarily placed between the shell 12 and the heating iron during heating. In either case, the heat is transferred through the shell 12 and into the underlaying layer of hotmelt glue which melts, becomes sticky, and adheres the adjacent layers to each other when it cools. When the heating iron is removed, the glue layer 24A-E will cool and securely bond the shell 12 to the fabric layer 26A, B and if hotmelt glue layer 24A-E was applied to both sides of the fabric layer 26A, B, it will be securely bonded to the foam layer 14 as well. Heat may be applied by a heated iron, a heated platen like a “tee shirt press,” heated air or any other heating means. This technique is inexpensive, vastly simplifies the manufacturing process of the mattress pad 10, allows internal reinforcement of the shell 12, remains flexible, makes no sticky adhesive mess, adheres in seconds and is not visible by external inspection of the shell 12.

Stabilizing the bottom side of the shell 12 by hotmelt gluing the shell 12 material to the foam 14 or to the fabric layer 26A, also makes the closing of the slit-like opening 18 much easier. Heat welding a strip of shell polymer 20 material across the slit-like opening 18 to seal the opening shut can be slow and difficult if the adjacent edges of the slit-like opening 18 are not stabilized. When the adjacent edges of the slit-like opening 18 are stabilized proximate each other by adhesively glueing them to the underlaying foam 14 or to the fabric layer 26A, closure of the slit-like opening 18 with a heat welded strip of shell polymer 20 material is fast and simple.

In some examples, EVA hotmelt glue is the preferred adhesive for attaching the layer of fabric to the shell and/or the layer of fabric to the layer of foam. It should be noted that other adhesives, pressure sensitive adhesives (PSA) and other hotmelt glue adhesives are contemplated.

Surgical table mattresses are traditionally attached to the top of the surgical table by a strip of hook and loop fasteners (e.g., Velcro) running along a longitudinal midline. In some examples, reinforcing the shell 12 of the bottom side of the mattress 10 with the fabric layer 26A and hotmelt glue layer 24A, B, vastly increases the strength and durability of the shell 12 for resisting damage when the hook and loop fastener that is typically attached to the shell 12 of the lower side of the mattress 10 is pulled apart from the surgical table-top for cleaning. The considerable force required to detach the hook and loop fasteners can damage the thin shell material over time.

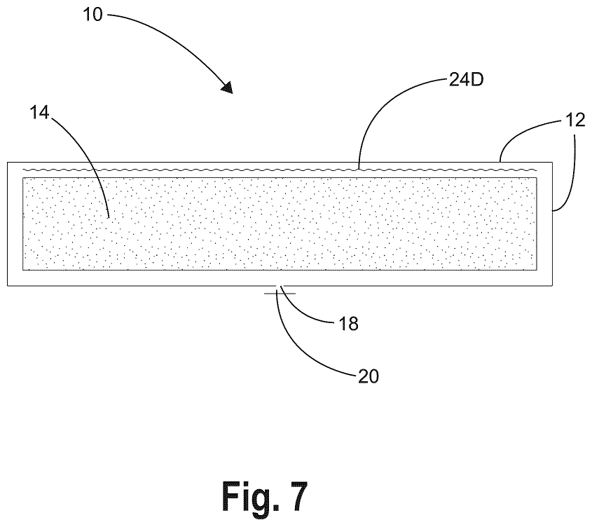

In some examples attaching at least a portion of the upper surface of the mattress 10 to an underlaying piece of fabric layer 26B or foam layer 14 can significantly increase the durability of the upper surface in resisting damage to the shell 12 that can occur during patient positioning. In some examples, especially in the case where the mattress 10 is going to be tipped into a steep Trendelenburg (head down) position, it may be advantageous to adhesively secure some or all of the shell 12 on the top side of the mattress 10 to the foam layer 14 or any fabric layer 26B that has been interposed, in order to prevent the fabric layers from slipping on each other when gravity tries to pull the patient down the incline. Maintaining the pressure off-loading enhancement of avoiding adhesively attaching the shell 12 to the entire top surface of foam layer 14 can be achieved at least in part by partially adhering the layers. For example, as shown in FIGS. 7-8, a longitudinal strip of hotmelt adhesive 24D that is 4-10″ wide may be applied along the longitudinal midline, which will stabilize the shell 12 with respect to the foam layer 14. Other widths and shapes of hotmelt adhesive 24D are contemplated. This longitudinal strip of hotmelt adhesive 24D may be protective from the longitudinal forces exerted by the weight of a patient in the Trendelenburg position causing sliding between the foam layer 14 and the shell 12, while simultaneously allowing the shell 12 to pull from the side edges toward the midline as the patient sinks into the mattress, minimizing “hammocking.” Limiting the adhesive bond to a strip down the midline allows the shell to freely pull in from the edges, relative to the foam layer 14 that it is bonded to along the midline. In some examples, the shell 12 may be adhesively bonded to the foam layer 14 using pressure sensitive adhesives (PSA), hot-melt adhesives, air cured adhesives or UV cured adhesives. Other adhesives and attachment techniques are contemplated.

As previously mentioned, the mattress pad 10 is typically attached to the surgical tabletop with a hook and loop fastener (e.g., Velcro) 28, as shown in FIG. 3. This can be problematic for three reasons. The significant purchase by the hook and loop fasteners that is needed to prevent the mattress pad 10 from moving on the tabletop 34, can result in damage to the shell 12 material when the pad 10 is repeatedly removed from the tabletop 34 for cleaning between surgical cases. Second, the hook and loop fasteners 28 are nearly impossible to clean when blood and other surgical contaminates get into the little spaces between the hook and loops. Finally, the hooks can break off with repeated use and eventually there are not enough left to secure the pad 10 to the tabletop 34 resulting in a failure to secure the mattress.

In some examples, it might be advantageous to use a different technology than hook and loop fasteners 28 for securing the surgical table pad or mattress 10 to the surgical tabletop 34. It should be understood that the primary force applied to the surgical table pad 10 during regular use is a sliding force, not a force trying to lift the pad 10 from the table. Hook and loop fasteners 28 provide much more resistance to lifting than is necessary to prevent sliding, and that is why they can damage the shell 12 during the frequent separation of the hook and loop fasteners 28 from the tabletop 34 for cleaning.

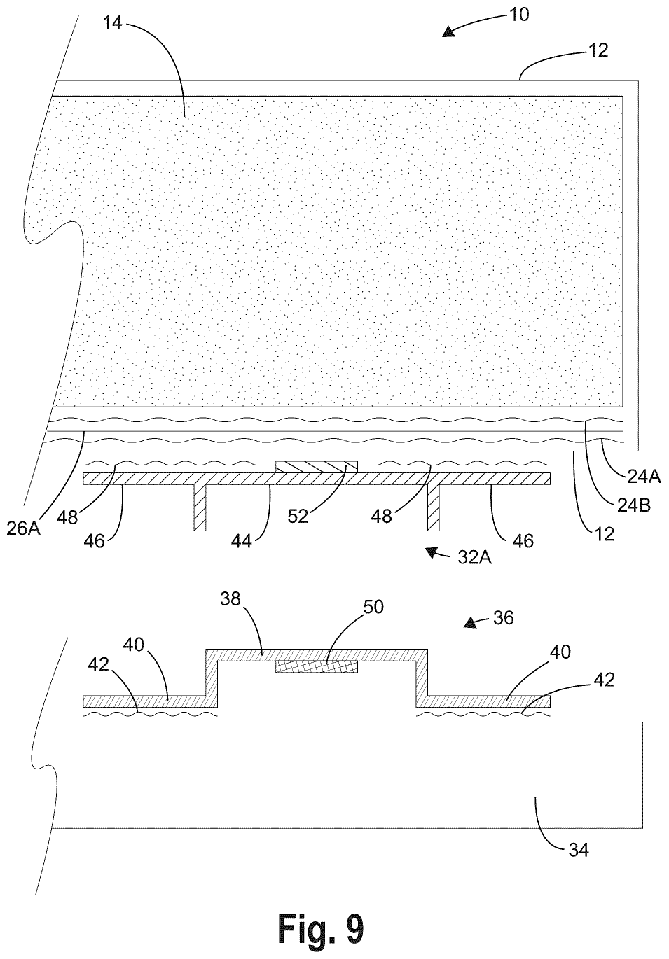

In some examples, it may be advantageous to replace the hook and loop fasteners 28 with four or more “post and cup” fasteners. As shown in FIG. 2, the four cup fastener components 32A-D of the post and cup fasteners may be located under the mattress 10, attached to the shell 12 near the sides and generally near the corners. Additional post and cup fasteners may be located under the mattress 10, attached to the shell 12 along the sides and between the four post and cup fasteners in the corners. FIG. 9 is a cross section taken through mattress 10 at 30-30 in FIG. 2 with the addition of a surgical tabletop 34.

In some examples, post and cup fasteners comprise a molded plastic post fastener component 36 that may be 0.25-0.5 in. height and 1-4 in. in diameter. Other heights, shapes (squares, hexagons, octagons etc.) and diameters are contemplated. In some examples, the post fastener component 36 includes an enclosed top 38 for strength and to prevent ingress of fluids for easy cleaning. In some examples, the post fastener component 36 also includes a surrounding flange 40 that can be adhesively 42 attached to the top of the surgical table 34.

In some examples, post and cup fasteners comprise a molded plastic cup component 32A-D that may be the same shape with the cup being slightly larger in internal diameter than the matching outer diameter of the post component 36. The molded plastic cup component 32A-D may be roughly the same height as the matching post component 36. The molded plastic post fastener component 36 is sized and shaped to fit inside the molded plastic cup component 32A-D in a “mated” fashion. In some examples, the cup component 32A-D may be closed on the side 44 facing the mattress shell 12 and may or may not include a surrounding flange 46, both of which can be adhesively 48 attached to the bottom side of the mattress shell 12. The cup fastener component 32A can be mated with or fit over the top of the matching post fastener component 36, preventing any sliding movement of the mattress 10 against the tabletop 34. The low profile of the post and cup fastener is imperceptible to touch under the 2-4-inch-thick foam compressible material layer 14.

The molded plastic cup component 32A-D and the molded plastic post component 36 may be made from many different moldable plastics including but not limited to: nylon, PVC and urethane.

In some examples, a magnet 50 may be attached under the closed surface 38 of the post component 36 and a flat piece of iron 52 attached under the closed surface 44 of the cup component 32A. Preferably attached in opposing locations, the magnet 50 and piece of iron 52 can magnetically engage with each other through the plastic layers of the closed central sections of the post 36 and cup 32. In this location, the magnet 50 and piece of iron 52 are secured within the post 36 and cup 32 components, are hidden from view and protected from liquid contamination. The magnetic force between the magnet 50 and piece of iron 52, is adequate to keep the cup fastener component 32A-D seated over the post fastener component 36 during normal use and it is still relatively easy to disengage the cup fastener component 32A-D from the post fastener component 36 while removing the mattress 10 from the surgical tabletop 34 for cleaning. Unlike the gradual fracture and failure of hooks on the hook and loop fasteners 28 with repeated use, the magnet 50 and piece of iron 52 retain a stable magnetism over time. It is contemplated that the location of the magnet 50 and piece of iron 52 could be reversed with the piece of iron 52 attached under the closed surface 38 of the post fastener component 36 and a magnet 50 attached under the closed surface 44 of the cup fastener component 32A-D. In some examples two magnets 50 and no iron 52 could be used. Other methods of attaching the cup fastener component 32A-D to the post fastener component 36 are contemplated, including but not limited to barb securement, ratchet securement and squeeze-fit securement.

An advantage of the surgical mattress securement system of this disclosure is that it can be easily retrofitted onto legacy surgical tabletops 34, in the field. In some examples, the post fastener components 36 can be mated with or fitted into the matching cup fastener components 32A-D, that are attached to the bottom of the mattress 10 in the factory, engaging the magnet 50 with the iron 52 to hold the two components together while positioning the post fastener components 36 for adhesion to the tabletop 34. In some examples, the release liners covering the pressure sensitive adhesive (PSA) 42 that is attached to the flanges 40 of the post fastener components 36 in the factory, can then be removed exposing the PSA. The mattress 10 can then be placed in its proper position on the surgical table 34. The post fastener components 36 that have been mated with or inserted into their matching cup fastener components 32A-D, can adhesively 42 attach to the tabletop 34 and will be automatically aligned with their matching cup fastener components 32A-D. The ability to easily and reliably retrofit this new technology to legacy surgical tables already installed in the operating room, provides a significant advantage for installation. Other adhesives and attachment means are contemplated for attaching the post fastener components 36 to the surgical tabletop 34. In some examples, heat, RF or ultrasound welds may be used to attach the molded cup fastener components 32A-D to the shell 12 material.

Unlike hook and loop fasteners 28, a further advantage of the post and cup surgical mattress securement system of this disclosure is that it provides tremendous resistance to the mattress 10 sliding on the upper surface of the surgical table 34 and yet is relatively easy to disengage from the table 34 for cleaning. A further advantage of the post and cup surgical mattress securement system of this disclosure is that it has no hidden recesses that can retain infectious material, is easy to clean and does not allow any fluid ingress. A further advantage of the post and cup surgical mattress securement system of this disclosure is that it easy to manufacture. A further advantage of the post and cup surgical mattress securement system of this disclosure is that it does not require cutting holes in the mattress shell 12 to accommodate the post. A further advantage of the post and cup surgical mattress securement system of this disclosure is that it does not require heat, RF or ultrasound welds to attach the molded cup fastener components 32A-D to the shell 12 material.

The post and cup surgical mattress securement system of this disclosure has been described as it may be used to secure the torso mattress pad to the surgical table but should be understood that this securement system can be used for securing any of the surgical table mattress pads 10. It is also apparent that the shell 12 attached to the molded plastic cup component 32A-D will be much stronger and more durable if constructed as shown in FIG. 9 and discussed above, with hotmelt glue adhering the shell 12 to either a reinforcing layer 26A or the foam layer 14.

Auscultation of breath sounds and other sounds from the chest is routinely accomplished by placing a stethoscope on the patient's back, over the lungs and listening. This is difficult in the operating room because most patients are operated on in the supine position, laying on their back. Auscultation of gross breath and heart sounds from the anterior chest and/or neck has been a standard monitor during anesthesia for the past 100 years. With the advent of electronic monitors such a pulse oximetry and end-tidal CO₂, clinicians have relied less and less on auscultation of gross breath and heart sounds, and many have abandoned routine auscultation all together.

Operating rooms are noisy environments and auscultation of subtle sounds with a standard stethoscope is nearly impossible due to the significant background noise and the limitations of human hearing. As a result, auscultation of any sounds other than gross breath and heart sounds in the operating room, has been impractical.

Microphones have been added to standard stethoscopes in order to amplify the sounds, but they amplify all sounds including the background noise. Simple electronic amplification of auscultated sounds has not been very useful.

Most surgery is done in the supine or lithotomy position with the patient's back laying on the mattress 10. Since operating tables are essentially the same size as the adult patient, in most cases it is predictable where the patient's chest will be located relative to the table. In some cases, the patient may be positioned toward either the foot or head ends of the table. In some cases, the patient may be positioned laterally (on their side) or supine (on their backs).

In some examples as shown in FIGS. 1 and 4, one or more microphones 54A, B may be placed on or in the mattress 10 of this disclosure and positioned where the patient's lungs are anticipated to be located on the mattress pad 10, lateral to the longitudinal midline of the mattress. In some examples as shown in FIG. 4, the microphones 54A, B may be located between the shell 12 material and the foam layer 14 of the mattress pad 10 of this disclosure. With only a thin layer of shell 12 material and maybe a bedsheet separating the microphones 54A, B from the skin of the patient's back, the one or more microphones 54A, B may be perfectly positioned to receive high-quality sounds from the patient's chest, through the back.

In some examples as shown in FIG. 5, one or more microphones 54A, B may be placed on the upper surface of the shell 12 material of the mattress pad 10 of this disclosure and positioned where the patient's lungs are anticipated to be located on the mattress pad 10. Microphones 54A, B on the upper surface of the pad 10 may be covered with a thin layer of polymeric film 56A, B such as urethane or PVC, for protection. The thin layer of polymeric film 56A, B may produce a better sound quality than placing the microphones 54A, B under the shell 12 material because the shell 12 material typically includes a layer of fabric. In some examples, one or more microphones 54A, B may be placed on the upper surface of the shell 12 material of the mattress pad 10 of this disclosure may be in their own protective shell such as a plastic coating.

In some examples, the 2-4 in. of foam in the foam layer 14 of the mattress 10 and a surgical tabletop 34 under the one or more microphones 54A, B, uniquely provides excellent sound deadening of the background noise. If the background noise can be deadened, then the subtle breath sounds and other sounds emanating from the chest can be detected by the one or more microphones 54A, B, amplified electronically and recorded. Auscultation of the posterior chest with the microphones 54A, B of this disclosure, with the muffling and deadening of the background noise by the mattress 10 and surgical tabletop 34, is diametrically different than attempting to listen to the anterior chest of a patient in a noisy operating room.

While auscultation with microphones 54A, B have been disclosed in surgical table mattresses or pads 10, we are also disclosing the placement of microphones 54A, B in any bed mattress. The position of a patient on a surgical table mattress 10 is predictable because the tables and mattresses 10 are sized to fit the average adult patient. In contrast, beds come in sizes that are much larger than the patient and therefore, the microphones 54A, B may not be positioned proximate the patient's chest. In this case, it may be advantageous to mount four or more microphones 54A, B across the surface of the bed so that at least one of them is positioned proximate a patient's lungs (e.g., in contact with the patient's chest, such as the patient's posterior chest). AI can easily determine which microphones 54A, B are not contacting the patient's chest or are listening to background noises (not contacting the patient at all) and simply ignore the input from those microphones 54A, B.

In some examples, the electronically recorded breath sounds may be analyzed with artificial intelligence (AI) or machine learning (ML). In contrast to the humans hearing subtle, individual sounds that are all jumbled together as a single sound, the computer using AI can separate the jumbled sounds into their individual components. This is similar to the sound of an orchestra at a point in time sounding like a single sound to the human ear. In contrast, the computer with AI can separate the sound into its components of individual instruments. The separation of sounds may occur based on one or more of frequency, amplitude and/or periodicity. Other identifiable features of the sounds including but not limited to harmonics are also contemplated for isolation and identification of the individual sound components. Because the sounds can be recorded, AI can look for subtle changes in a given sound over time, whereas the human ear could never discern subtle changes over time.

In some examples, breath sounds may be monitored to document successful tracheal intubation and ventilation. Breath sounds may be monitored to document spontaneous ventilation. In some examples, wheezing emanating from large airways may be separated from wheezing emanating from small airways, where the treatment may be different. Breath sound symmetry between the two sides of the chest may be monitored to detect tracheal tube misplacement or pneumothorax.

Breath sounds may be monitored and used for “sensor fusion” validation of ventilation. For example, pulse oximetry may indicate adequate ventilation for a period of time even after a lack of ventilation (it takes a short while to desaturate), which could be detected instantly by auscultation. The sound of the heart beating may be monitored and used for “sensor fusion” validation of the electrocardiogram (ECG) trace. A loose ECG lead can make the ECG trace look like ventricular fibrillation (V-fib). V-fib can quickly be ruled out if a rhythmic “lub-dub” is heard with auscultation. Humans are trained to evaluate all problems by comparing the output of more than one monitor, a form of sensor fusion. Computers need a formal system for ignoring faulty monitor data and that is known as “sensor fusion.” The auscultation with AI analysis of this disclosure uniquely produces a number of useful sensor fusion data inputs.

Heart sounds including subtle murmurs can be monitored for changes over time indicating changes in blood flow. The patient's aorta is located very close to microphones 54A, B and blood flow through the aorta can be easily heard. In some examples, the sound of blood flowing through the major arteries such as the aorta may be correlated with cardiac output. An absolute cardiac output number may not be determinable but relative cardiac outputs over time may be determined. Other hemodynamic variables may be determinable from the sounds of blood flowing.

“Rales” is the effervescent sound emanating from wet alveoli popping open during inspiration. Rales are very subtle and cannot be heard by standard auscultation in an operating room environment. Rales are difficult to hear but AI can not only hear them but grade them in severity, if the background noise is deadened. Rales typically occur in the most dependent (lower) parts of the lung. Placing the microphones 54A, B in the mattress 10, uniquely positions the microphones 54A, B directly against the most dependent part of the lung when the patent is laying supine on the mattress 10. This is unique in that routine auscultation of the back of a person who is in a vertical position, does not allow the microphone or stethoscope to be directly adjacent the most dependent surface of the lung.

Generally, rales in a vertical patient are regarded as a sign of hypervolemia (high blood volume) or heart failure. However, in some examples mild rales in the dependent lung directly next to the microphones 54A, B of a supine patient may indicate normal volume status. Gross rales may indicate hypervolemia (high blood volume) or heart failure, and no rales from the most dependent lung may indicate hypovolemia (low blood volume). Auscultation with AI listening for dependent rales may be a patient blood volume status monitor that is currently not available for clinical use.

It is apparent that auscultation with deadening of the background noise by the mattress and AI interpretation of the subtle sound components can serve as a monitor for a wide variety of physiologic parameters, some of which are qualitative and some are quantitative. Further, subtle changes in any given sound may be detected over time and may indicate changes in physiologic status.

In the foregoing detailed description, the invention has been described with reference to specific embodiments. However, it may be appreciated that various modifications, changes and alternative combinations can be made without departing from the scope of the invention as set forth in the appended claims. Although embodiments of the invention are described in the context of a hospital operating room, it is contemplated that some embodiments of the invention may be used in other environments. Those embodiments of the present invention, which are not intended for use in an operating environment and need not meet stringent FDA requirements for repeated used in an operating environment, need not including particular features described herein. Thus, some of the features of preferred embodiments described herein are not necessarily included in preferred embodiments of the invention which are intended for alternative uses.

1. A method for manufacturing a reinforced surgical table mattress or pad, the method comprising:
inserting a layer of polymeric foam into a shell via a slit-like opening in the shell, the shell comprising one or more sheets of flexible polymeric film material laminated to a fabric, the one or more sheets of flexible polymeric film material being welded together at one or more of their edges to make a water-proof shell having a top layer and a bottom layer that is formed to surround the layer of polymeric foam;
inserting at least one layer of hotmelt glue film through the slit-like opening and so that the at least one layer of hotmelt glue film is positioned inside the shell between a bottom side of the layer of polymeric foam and the fabric of the bottom layer of the shell material; and
applying heat that is above the melting temperature of the hotmelt glue film and below the melting temperature of the polymeric film shell material to the outside of the bottom of the shell, melting the at least one layer of hotmelt glue film and bonding the fabric of the bottom layer of shell material to an adjacent layer of material when the hotmelt glue film cools.

2. The method for manufacturing the reinforced surgical table mattress or pad of claim 1, further comprising welding the one or more sheets of flexible polymeric film, wherein the welding is one or more of a heat weld, radio frequency (RF) weld, or ultrasound weld.

3. The method for manufacturing the reinforced surgical table mattress or pad of claim 1, wherein the layer of hotmelt glue film adheres the fabric of the bottom layer of the shell material to the bottom side of the layer of polymeric foam.

4. The method for manufacturing the reinforced surgical table mattress or pad of claim 1, wherein the layer of hotmelt glue film adheres the fabric of the bottom layer of the shell material to a layer of reinforcing fabric.

5. The method for manufacturing the reinforced surgical table mattress or pad of claim 1, wherein a layer of hotmelt glue film adheres the fabric of the bottom layer of the shell material to a layer of reinforcing fabric and a second layer of hotmelt glue film adheres the layer of reinforcing fabric to the bottom side of the layer of polymeric foam.

6. The method for manufacturing the reinforced surgical table mattress or pad of claim 1, wherein an additional layer of hotmelt glue film adheres some or all of the fabric of the top layer of the shell material to a layer of reinforcing fabric or to the top side of the layer of polymeric foam.

7. The method for manufacturing the reinforced surgical table mattress or pad of claim 1, further comprising welding a strip of shell polymer over the slit-like opening in the bottom layer of shell material, and wherein the layer of hotmelt glue film that bonds the fabric of the bottom layer of shell material and to the adjacent layer of material also stabilizes the fabric of the bottom layer of shell material during the process of welding the strip of shell polymer over the slit-like opening in the bottom layer of shell material.

8. The method for manufacturing the reinforced surgical table mattress or pad of claim 1, further comprising mounting two or more microphones on an upper surface of the top layer of the shell material on each side of a longitudinal midline and in a region corresponding with a location that supports a patient's chest.

9. The method for manufacturing the reinforced surgical table mattress or pad of claim 8, further comprising separating, analyzing, and interpreting the components of the sound emanating from the patient's chest using artificial intelligence (AI) or machine learning (ML).

10. The method for manufacturing the reinforced surgical table mattress or pad of claim 1, further comprising mounting two or more microphones below the top layer of the shell material on each side of a longitudinal midline in a region corresponding with a location that supports a patient's chest.

11. The method for manufacturing the reinforced surgical table mattress or pad of claim 10, further comprising separating, analyzing, and interpreting the components of the sound emanating from the patient's chest using artificial intelligence (AI) or machine learning (ML).

12. The method for manufacturing the reinforced surgical table mattress or pad of claim 1, further comprising:
adhesively attaching four or more molded plastic cups to the bottom side of the mattress or pad; and
securing the mattress or pad to a surgical table by the four or more molded plastic cups by mating the four or more cups with four or more plastic posts that are adhesively attached to a surgical tabletop.

13. The method for manufacturing the reinforced surgical table mattress or pad of claim 12, further comprising inserting a magnet or a piece of iron in the molded plastic cups to mate with a piece of iron or magnet in the posts.

14. A reinforced surgical table mattress or pad comprising:
a layer of polymeric foam; and
a shell covering the layer of polymeric foam and comprising one or more sheets of flexible polymeric film material laminated to a fabric; and
the one or more sheets of flexible polymeric film material are welded together at one or more of their edges to make a water-proof shell having a top layer and a bottom layer and that is formed to surround the layer of polymeric foam; and
a slit-like opening in the shell on a bottom side or an end side allowing for the insertion of the layer of polymeric foam into the welded shell; and
an at least one layer of hotmelt glue film that has been applied to a layer of reinforcing fabric is positioned inside the shell between a bottom side of the layer of polymeric foam and the fabric of the bottom layer of the shell material; wherein
the at least one layer of hotmelt glue film is adhered to the fabric of the bottom layer of shell material and to the layer of reinforcing fabric by applying heat to the outside of the bottom of the shell, melting the hotmelt glue film and then letting the hotmelt glue film cool; and
the temperature of the applied heat is above the melting temperature of the hotmelt glue film and below the melting temperature of the polymeric film shell material.

15. The reinforced surgical table mattress or pad of claim 14, wherein the weld is one or more of a heat weld, radio frequency (RF) weld, or ultrasound weld.

16. The reinforced surgical table mattress or pad of claim 14, wherein a second layer of hotmelt glue film adheres the layer of reinforcing fabric to the bottom side of the layer of polymeric foam.

17. The reinforced surgical table mattress or pad of claim 14, wherein an additional layer of hotmelt glue film adheres some or all of the fabric of the top layer of the shell material to a second layer of reinforcing fabric or to the top side of the layer of polymeric foam.

18. The reinforced surgical table mattress or pad of claim 14, further comprising a strip of shell polymer over the slit-like opening, and wherein the layer of hotmelt glue film that bonds the fabric of the bottom layer of shell material and to an adjacent layer of material also stabilizes the fabric of the bottom layer of shell material during the process of welding a strip of shell polymer over the slit-like opening in the bottom layer of shell material.

19. The reinforced surgical table mattress or pad of claim 14, wherein two or more microphones are mounted on the upper surface of the top layer of the shell material on each side of a longitudinal midline in a region corresponding with a location that supports the patient's chest.

20. The reinforced surgical table mattress or pad of claim 19, wherein the components of the sound emanating from the patient's chest are separated, analyzed and interpreted using artificial intelligence (AI) or machine learning (ML).

21. The reinforced surgical table mattress or pad of claim 14, wherein two or more microphones are mounted below the top layer of the shell material on each side of a longitudinal midline in a region corresponding with a location that supports the patient's chest.

22. The reinforced surgical table mattress or pad of claim 21, wherein the components of the sound emanating from the patient's chest are separated, analyzed and interpreted using artificial intelligence (AI) or machine learning (ML).

23. The reinforced surgical table mattress or pad of claim 14, wherein the mattress or pad comprises four or more molded plastic cups that are adhesively attached to the bottom side of the mattress or pad and configured so that the mattress or pad can be secured to the surgical table by mating the four or more cups with four or more plastic posts that can be adhesively attached to the surgical tabletop.

24. The reinforced surgical table mattress or pad of claim 23, wherein four or more molded plastic cups comprises a magnet or a piece of iron configured to mate with a magnet or piece of iron in the posts.

25. A method for manufacturing a reinforced surgical table mattress or pad, the method comprising:
providing a surgical table mattress or pad comprising:
a layer of polymeric foam; and
a shell covering the layer of polymeric foam and comprising one or more sheets of flexible polymeric film material laminated to a fabric; and
the one or more sheets of flexible polymeric film material are welded together at one or more of their edges to make a water-proof shell having a top layer and a bottom layer and that is formed to surround the layer of polymeric foam; and
a slit-like opening in the shell on a bottom side or an end side allowing for the insertion of the layer of polymeric foam into the welded shell; and
positioning a layer of reinforcing fabric having at least one layer of hotmelt glue film that has been applied to the layer of reinforcing fabric inside the shell between a bottom side of the layer of polymeric foam and the fabric of the bottom layer of the shell material;
adhering the at least one layer of hotmelt glue film to the fabric of the bottom layer of shell material and to the layer of reinforcing fabric by applying heat to the outside of the bottom of the shell, melting the hotmelt glue film and then letting the hotmelt glue film cool; and wherein
the temperature of the applied heat is above the melting temperature of the hotmelt glue film and below the melting temperature of the polymeric film shell material.

26. The method for manufacturing a reinforced surgical table mattress or pad of claim 25, further comprising welding the one or more sheets of flexible polymeric film, wherein the welding is one or more of a heat weld, radio frequency (RF) weld, or ultrasound weld.

27. The method for manufacturing a reinforced surgical table mattress or pad of claim 25, wherein a second layer of hotmelt glue film adheres the layer of reinforcing fabric to the bottom side of the layer of polymeric foam.

28. The method for manufacturing a reinforced surgical table mattress or pad of claim 25, wherein an additional layer of hotmelt glue film adheres some or all of the fabric of the top layer of the shell material to a second layer of reinforcing fabric or to the top side of the layer of polymeric foam.

29. The method for manufacturing a reinforced surgical table mattress or pad of claim 25, further comprising welding a strip of shell polymer over the slit-like opening in the bottom layer of shell material, and wherein the layer of hotmelt glue film that bonds the fabric of the bottom layer of shell material and to an adjacent layer of material also stabilizes the fabric of the bottom layer of shell material during the process of welding the strip of shell polymer over the slit-like opening in the bottom layer of shell material.

30. The method for manufacturing a reinforced surgical table mattress or pad of claim 25, further comprising mounting two or more microphones on an upper surface of the top layer of the shell material on each side of a longitudinal midline and in a region corresponding with a location that supports a patient's chest.

31. The method for manufacturing a reinforced surgical table mattress or pad of claim 30, further comprising separating, analyzing, and interpreting the components of the sound emanating from the patient's chest using artificial intelligence (AI) or machine learning (ML).

32. The method for manufacturing a reinforced surgical table mattress or pad of claim 25, further comprising mounting two or more microphones below the top layer of the shell material on each side of a longitudinal midline in a region corresponding with a location that supports a patient's chest.

33. The method for manufacturing a reinforced surgical table mattress or pad of claim 32, further comprising separating, analyzing, and interpreting the components of the sound emanating from the patient's chest using artificial intelligence (AI) or machine learning (ML).

34. The method for manufacturing a reinforced surgical table mattress or pad of claim 25, further comprising:
adhesively attaching four or more molded plastic cups to the bottom side of the mattress or pad; and
securing the mattress or pad to a surgical table by the four or more molded plastic cups by mating the four or more cups with four or more plastic posts that are adhesively attached to a surgical tabletop.

35. The method for manufacturing a reinforced surgical table mattress or pad of claim 34 further comprising inserting a magnet or a piece of iron in the molded plastic cups to mate with a piece of iron or magnet in the posts.

36. A reinforced surgical table mattress or pad comprising:
a layer of polymeric foam; and
a shell covering the layer of polymeric foam and comprising one or more sheets of flexible polymeric film material laminated to a fabric; and
the one or more sheets of flexible polymeric film material are welded together at one or more of their edges to make a water-proof shell having a top layer and a bottom layer and that is formed to surround the layer of polymeric foam; and
a slit-like opening in the shell on a bottom side or an end side allowing for the insertion of the layer of polymeric foam into the welded shell; and
an at least one layer of hotmelt glue film positioned inside the shell between a bottom side of the layer of polymeric foam and the fabric of the bottom layer of the shell material; wherein
when heat that is above the melting temperature of the hotmelt glue film and below the melting temperature of the polymeric film shell material is applied to the outside of the bottom of the shell, the at least one layer of hotmelt glue film melts; and
when the at least one layer of the hotmelt glue film cools, it bonds the fabric of the bottom layer of shell material to an adjacent layer of material.
